(19) United States
(12) Patent Application Publication
(10) Pub. No.: US 2020/0008929 A1
(43) Pub. Date: Jan. 9, 2020

(54) MEDICAL DEVICE FOR BREAST SURGERY

(71) Applicant: DECO MED SRL, Marcon (IT)

(72) Inventor: Giovanni BERTOLI, Marcon (IT)

(73) Assignee: DECO MED SRL, Marcon (IT)

(21) Appl. No.: 16/492,287

(22) PCT Filed: Mar. 5, 2018
(86) PCT No.: PCT/IT2018/050034

§ 371 Date: Sep. 9, 2019

(30) Foreign Application Priority Data
Mar. 9, 2017 (IT) 102017000025987

(51) Int. Cl. A61F 2/12; A61L 27/36

(52) U.S. Cl. CPC A61F 2/12; A61L 27/3633; A61F 2210/0014; A61L 2400/16; A61L 27/24; A61F 2230/0071; A61F 2230/0073; A61F 2250/0039; A61L 2430/04

(57) ABSTRACT

A device is initially rolled up can unfold into its original spherical dome shape after insertion through a surgical incision.

The invention described in this patent application is a device that is initially rolled up and can unfold into its original spherical dome shape after insertion through a surgical incision.

The invention described in this patent application can be specifically used in the framework of additive mastoplasty.

A complication called “bottoming-out” is known to result from additive mastoplasty. When the breast prosthesis is positioned by the surgeon between the mammary gland and the greater pectoral muscle, there is a risk of the prosthesis sliding under the submammary groove.

The reasons for the so-called “bottoming-out” process could be the weight and unnatural volume of the prosthesis that cannot be supported by tissues of the groove, or its failure to remain within that anatomical line due to the surgeon's creation of a pocket that is too large.

When the surgeon places the breast prosthesis under the greater pectoral muscle, the above complication manifests more rarely.

Indeed, it can be theorised that, since the greater pectoral muscle is firmly anchored by a tendon to the thoracic ribs, it does not give way under the weight of a breast prosthesis and the tension of an unnatural volume.

The aesthetic effect of the “bottoming-out” process reveals breast volume below its natural position, whereas the “areola-nipple” complex remains in its anatomical site due to sagging of internal tissues in the submammary groove. Stressed by the weight and volume of a breast prosthesis, these tissues collapse and the breast prosthesis slips downwards through this space.

This complication necessarily requires revision surgery.

Many surgical techniques restore the submammary groove and the natural position of the breast prosthesis.

All these revision techniques envisage obliteration of tissues at the groove and their reinforcement with non-absorbable sutures to restore the correct position of the breast prosthesis (above the groove).

Hence, the ideal goal is to avoid resorting to the operating room to perform revision surgery on sagging tissues of the submammary groove resulting from the unnatural weight and volume of a silicone prosthesis positioned for additive purposes prevalently between the greater pectoral muscle and the mammary gland.

Modern techniques exploit the elasticity of ECM biomaterial.

Once it is hydrated, it can be bent and subjected to traction to better adapt to its function inside the body.

It can also be cut out to improve adaptation. It can be sutured to human tissue or between flaps of the same.

The industrialisation process of this ECM biomaterial has led to the innovative production of multiple medical devices, all of which have in common a flat geometrical shape.

By applying a defined axial load, besides hydration, dehydration and mechanical pressing processes, flat sheets of the extracellular matrix (ECM) are moulded into geometrical 3D solids derived from the sphere, thus endowing them, if they are finally dehydrated, with a specific shape and memory of shape.

Indeed we must consider that the extracellular matrix (ECM) is made up of dense parallel fascia of collagen fibres, while elastin tissue fascia have been destructured by the deantigenation process.

By expelling water and air from the extracellular matrix, these collagen fibres can be compressed to the point of undergoing a plastic deformation that determines the new shape. Resorting to the use of extracellular matrices (ECM) is not a rare event in the field of breast surgery. As a result of their regenerative properties, these matrices reinforce tissues with which they are placed in contact.

The membranes of the extracellular matrix (ECM) can be recognised as “self” by the body because they are made up of collagen, a protein that is well preserved in mammals.

They are involved in the regenerative process implemented by the body following tissue damage generated by surgery. The body repairs damaged tissues and regenerates the collagen membrane as if it were damaged tissue.

This involvement results in the formation of a new vascularised fascia, which acts as complementary tissue that strengthens adjacent tissue.

Implantation of inert biomaterials (or of synthetic meshes) is allowed by the scientific community, despite the current evidence that such an implant (inert) does not trigger signs of recognition that activate the regenerative process in the host body.

Conversely, the biological process that is stimulated by an inert biomaterial is a defensive process. [1. James M. Anderson, Analiz Rodriguez, and David T. Chang. Foreign Body reaction to biomaterials. Semin Immunol, 2008; 20(2): 86, -100, 2. Anderson J M. Biological responses to materials. Annu. Rev. Mater. Res, 2001; 31: 81-110.] The body does not recognise this inert biomaterial as “self” and, therefore, it will be inclined to isolate it through the formation of stiff tissue that is palpable. Conversely, a bioactive biomaterial (ECM) will be recognised as “self” and the body will be inclined to incorporate it by regenerating it into “self” tissue. [3. Cornwell K G, Landsman A, James K S. Extracellular matrix biomaterials for soft tissue repair. Clin Podiatr Med Surg, 2009; 26(4):507-23.]

Isolation or regeneration mechanisms are, therefore, induced by the nature of the biomaterial that is implanted in the human body.

The cellular actors that perform these functions (isolation or regeneration) are initially the same.

In fact, fibroblasts are engaged by active growth factors whenever there is tissue damage. When there is an implant of inert biomaterial (synthetic mesh), fibroblasts are transformed into myofibroblasts, cells with elastic properties that can lengthen to include and isolate a foreign body from surrounding tissue. [2] Instead, when there is bioactive biomaterial (ECM), the same fibroblasts transform it into native tissue that is not palpable, with the subsequent presence of a scarce myofibroblastic population.

The biological matrix (ECM) is transformed into self-tissue (incorporation) in physical conditions of intimate contact between the biological matrix (ECM) and the vascularised tissue flap.

This intimate contact means that a larger number of interface points between matrix and vascularised tissue corresponds to a higher possibility of transforming the matrix into self-tissue. Hence, adaptation of the matrix to the implantation site is crucial to ensure an excellent interface for maximum contact.

If the implantation site were cylindrical, maximum contact between matrix and site would require a cylindrical matrix, just as a flat sheet would be required, if the implantation site were a flat surface.

We know that the geometrical shape of the breast can be traced to a geometrical solid derived from the sphere. As described above, maximum contact between matrix and implantation site requires a spherical or semi-spherical shape.

Today there are cylindrical medical devices or devices with a similar shape, as they are flat sheets that are later rolled up.

For instance, patent FR3025999 describes a rolled up, square or rectangular, protective implant. The solution described in document FR3025999 is designed to protect the spermatic cord. It presents as flat synthetic material, which has no memory of shape and is rolled up into the cylindrical shape of either a sleeve or a protective tube.

Moreover, document FR3025999 describes anchoring devices to maintain the shape required, hence to keep it rolled up during use.

Today we also have document AU2009288233, which describes a flat sheet that is rolled up to wrap and protect damaged cylindrical tissue.

The rolling up process allows the matrix to overlap. Hence, rolled up on itself, it forms a sort of multilayer that prevents cellular penetration.

Document KR20040030859 describes a medical device and its related system to close blood vessels inside the human body. It is a haemostatic device that blocks blood flow and absorbs the blood.

This too always presents with a flat shape. The device described in document KR20040030859 comprises a multilayer of various materials, particularly a layer of ECM and a layer of spongy or foamy material. This multilayer is rolled up by means of a stainless steel wire to form a tightly compressed layer that prevents blood flow and absorption.

Document U.S. Pat. No. 9,149,354 describes a device that repairs the flexor tendon. It is made up of a semi-rigid synthetic mesh with a continuous suture thread on two opposite sides. This thread allows to close the device by forming a cylindrical structure. Document US20140155917 describes a prosthetic device and its anchoring methods in the field of hernia repair. It is flat with rods placed on the flat surface (it is made of synthetic material, precisely polypropylene filaments).

The patent claims a system that includes a sheet of synthetic mesh and two or more removable rods secured to the synthetic mesh itself to enhance its stiffness.

Even document US20130282033 describes a kit made up of a sheet of synthetic material that is smooth on one side and adhesive on the other to allow vertical anchoring of the rods. Once set in place, the rods allow the flat sheet to be folded. These rods are removed once the device is inserted into the human body.

Patent application WO2011140382 describes a device that has been studied to insert a rolled up prosthesis that, once inserted through a surgical incision, unfolds at the site of insertion.

The scope of the invention described in patent application WO2011140382 is, therefore, to create a device for insertion of a rolled up implant that might not be inserted by itself without appropriate devices because it is not sufficiently stiff as it is originally a flat sheet.

The application for patent US2009149953 describes a rolled up prosthesis with irregular geometrical features presenting an inferior swelling on the anterior side, and superiorly a portion with decreasing thickness.

Hence, all known state-of-the-art solutions concern devices that have a cylindrical shape obtained by rolling up a flat matrix sheet, thus exploiting only the elasticity of the biomaterial to unfold it, if required.

The above solutions, which are known today, are studied to maintain this cylindrical shape even during actual use. They need securing elements, such as rings or rods, to maintain the cylindrical shape constant in time or to ensure the system's stiffness.

We know that a geometrical solid can be either a polyhedron or a solid of revolution.

The former is made up of a combination of flat figures, while the latter issues from the rotation of flat figures.

The polyhedron has flat surfaces (pyramid, cube, prism, etc.), while the solid of revolution derived from the sphere has curved lateral surfaces (sphere, hemisphere, spherical dome, spherical wedge, etc.).

Hence, the internal surface of the breast can be considered as the volume occupied by a complex solid of revolution derived from the sphere with curved lateral surfaces, hereinafter referred to as “spherical dome”. Subsequently, any element made up of a spherical dome can line the mammary chamber, maintaining intimate contact with it.

The purpose of the invention described in this patent application is, therefore, to create a medical device made up of ECM to reinforce the mammary groove and/or to obliterate dead spaces by adapting to the mammary groove itself. Hence, it resembles a spherical dome that, prior to implantation, is rolled up on itself to form a cylinder that can be inserted with appropriate devices, preferably but not solely an introducing ring, into the breast through the surgical incision. After insertion, this device can unfold into its original configuration of a spherical dome in the bottom end of a mammary pocket to reinforce the groove and/or to obliterate dead spaces.

The invention described in this patent application thus presents as a system created by rolling up the circular segment; hence, it has an oblong shape that is tapered on both ends.

This device is, therefore, restrained preferably but not solely by a retention ring and, once inserted into the human body, it opens and recovers its semi-spherical shape as a result of the memory of shape that characterises it.

The medical device examined is required to reinforce, with the first implant, the tissues of the submammary groove to avoid the “bottoming-out” complication, and hence the patient's return to the operating room. Obliteration of dead spaces performed with a biological matrix inhibits the deposit of serum. The “male” on “female” shape guarantees intimate contact.

The invention concerned by this patent application is illustrated in Tables 1 and 2 (enclosed), where:

Table 1/2

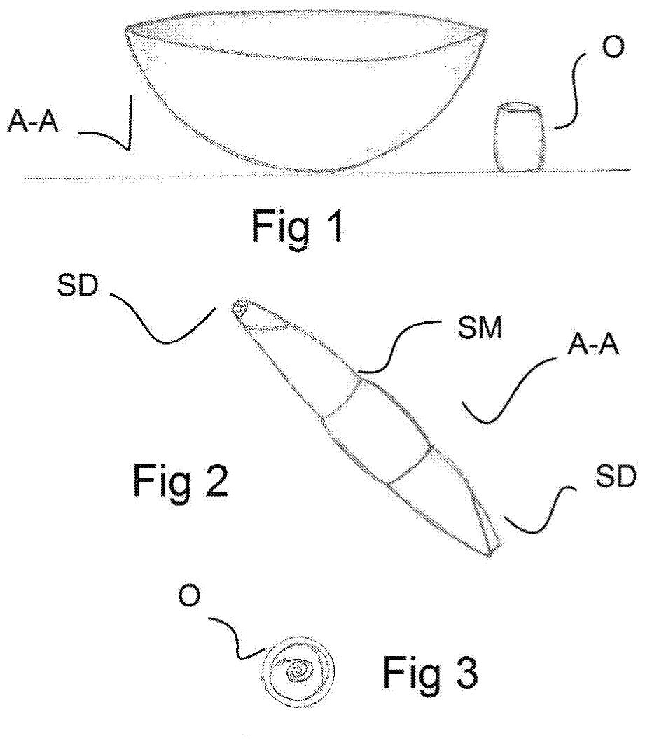

FIG. 1

C—Dome with ECM matrix

O—Restraining device

FIG. 2—Device concerned by the patent application.

SM—Median section

SD—Distal sections

FIG. 3—View of device section concerned by the patent application.

Table 2/2

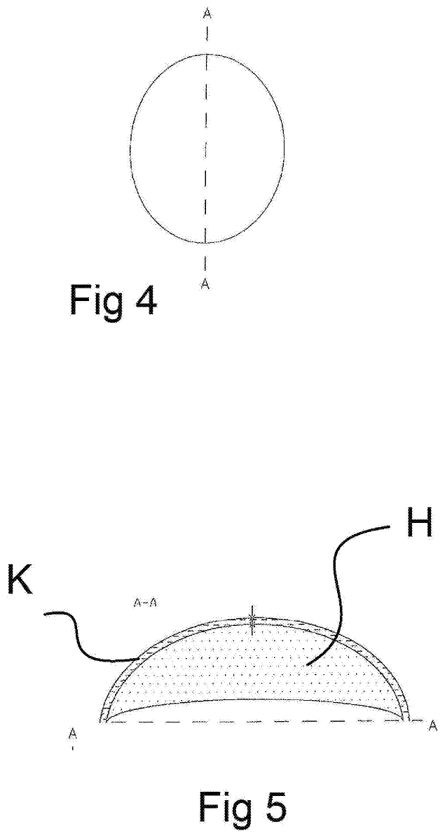

FIG. 4

A-A Spherical dome in ECM

FIG. 5

H—air volume

K—collagen

The invention concerned by this patent application is illustrated in FIG. 2. It presents as a cylindrical solid obtained by rolling a spherical dome onto itself (A-A FIG. 1,3,4,5). The median central section (SM) is larger than the distal sections (SD). Particularly the spherical dome (A-A) is made up of extracellular matrix (ECM) obtained by means of specific plastic deformation processing of a flat ECM sheet to obtain a curved square shape, in this case concave/convex with 3D development that can be traced to a lens-like shape.

Since the dome (A-A FIG. 1,4,5) has a memory of shape, once rolled up and inserted into the human body, it unfolds into a curved square shape (A-A FIG. 4), with 3D development resembling a lens-like shape.

In the conformation illustrated in FIG. 2, the device is introduced through a small surgical incision (4/6 cm), and on reaching the site, with its memory of shape it regains the convex dome shape that matches the concavity of the submammary groove, whose shape is also derived from the sphere.

A retention ring can be preferably but not solely placed at the centre to act as a guide for the device prior to insertion on site.

The retention device O, preferably but not solely a ring, has a dual function of maintaining the dome A-A rolled up to inhibit the memory of shape that is inclined to unfold dome A-A, and to facilitate introduction of the device through the surgical incision. Indeed, the retention device O acts as a guide to insert the device into the mammary groove.

The invention produced as described allows introduction of the device through a surgical incision that is a few centimeters long (range 2/6), to then regain its industrial shape of spherical dome A-A inside the breast, as it will not be restrained anymore by the retention device O. The dome will establish intimate contact with the mammary groove, whose shape results from the sphere. The above is studied to both reinforce the submammary groove and to avoid dead spaces between tissue structures.

Particularly the invention described in this patent application is made up of an active biomaterial (ECM), by way of non-exhaustive example, heterologous or homologous deantigenated dermis, heterologous or homologous deantigenated pericardium, heterologous or homologous deantigenated bladder or any other bioactive biomaterial (ECM) made of collagen.

Upon unfolding, the shape of the invention adapts to the floor of the groove, a spherical curve on a spherical curve, thus generating, through intimate contact, new reinforcing tissue and reducing the risk of the mammary prosthesis slipping under it.

1. A medical device for breast surgery said medical device originally constituted by a quadratic matrix of hemispherical or paraboloid or ellipsoid or hyperboloid shape, said quadratic matrix having a three-dimensional development, said matrix being rolled to obtain a solid of revolution said solid of revolution having a central section greater than the distal sections and said solid of revolution being tapered at the extremities.

2. Medical device according to claim 1 wherein it is equipped with a restraint device.

3. Medical device according to claim 1, wherein the quadratic matrix is made of collagen membrane having shape memory.

4. Medical device according to claim 1, wherein the restraint device is in biomaterial.

5. Medical device according to claim 1, wherein once inserted in a surgical wound the solid of revolution returns at the original hemispherical or paraboloid or ellipsoid or hyperboloid shape.

6. Medical device according to claim 2, wherein the quadratic matrix is made of collagen membrane having shape memory.

7. Medical device according to claim 2, wherein the restraint device is in biomaterial.

8. Medical device according to claim 3, wherein the restraint device is in biomaterial.

9. Medical device according to claim 6, wherein the restraint device is in biomaterial.

10. Medical device according to claim 2, wherein once inserted in a surgical wound the solid of revolution returns at the original hemispherical or paraboloid or ellipsoid or hyperboloid shape.

11. Medical device according to claim 3, wherein once inserted in a surgical wound the solid of revolution returns at the original hemispherical or paraboloid or ellipsoid or hyperboloid shape.

12. Medical device according to claim 4, wherein once inserted in a surgical wound the solid of revolution returns at the original hemispherical or paraboloid or ellipsoid or hyperboloid shape.

13. Medical device according to claim 6, wherein once inserted in a surgical wound the solid of revolution returns at the original hemispherical or paraboloid or ellipsoid or hyperboloid shape.

14. Medical device according to claim 7, wherein once inserted in a surgical wound the solid of revolution returns at the original hemispherical or paraboloid or ellipsoid or hyperboloid shape.

15. Medical device according to claim 8, wherein once inserted in a surgical wound the solid of revolution returns at the original hemispherical or paraboloid or ellipsoid or hyperboloid shape.

16. Medical device according to claim 9, wherein once inserted in a surgical wound the solid of revolution returns at the original hemispherical or paraboloid or ellipsoid or hyperboloid shape.
